(19) United States
(12) Patent Application Publication
(10) Pub. No.: US 2006/0165611 A1
(43) Pub. Date: Jul. 27, 2006

(54) Composition for Treating and Preventing Periodontal Disease and Method of Use

(75) Inventor: Gerald L. Maurer, Cincinnati, OH (US)

Correspondence Address: LAFKAS PATENT LLC, 7811 LAUREL AVENUE, CINCINNATI, OH, 45243, US

(73) Assignee: NATIONAL RESEARCH LABORATORIES, LTD., Cincinnati, OH (US)

(21) Appl. No.: 11/307,140

(22) Filed: Jan. 25, 2006

Related U.S. Application Data
(60) Provisional application No. 60/593,563, filed on Jan. 26, 2005.

(51) Int. Cl. A61K 8/35; A61K 8/19

(52) U.S. Cl. 424/49

(57) ABSTRACT

The present invention is a mixture for treating and preventing periodontal disease comprising acemannan, an immuno-stimulant with tissue repair activity and a hydrated dialkali monometal polycarboxylate 1:1 molar ratio of metal-to-complexing agent, such that lysyl oxidase for collagen formation and subsequent tissue repair is activated.

### CROSS-REFERENCE TO RELATED APPLICATION

This application for a patent claims priority to U.S. Provisional Patent Application No. 60/593,563 as filed Jan. 26, 2005.

### BACKGROUND

The present invention relates generally to a composition and method of using such composition to topically assist in healing and to substantially prevent periodontal disease. In particular, the composition comprises an immune system stimulant and an anti-inflammatory agent topically applied to treat and substantially prevent periodontal disease.

Periodontal disease, also known as gum disease, is a leading cause of tooth loss in adults. In fact, about 70 percent of adult tooth loss can be attributed to periodontal disease, and affects approximately three out of four persons at some point in their life.

Most periodontal disease is caused by bacterial plaque, which appears as a sticky, colorless film that forms on teeth. Different types of periodontal disease may be caused by differing types of bacteria. The bacterial plaque may harden into a rough, porous substance known as calculus or tartar. The plaque produces and expels toxins that irritate gums and eventually results in a breakdown of the fibers that hold the gums tightly to teeth.

As the fibers break down, periodontal pockets develop and fill with more bacteria and toxins, creating more and deeper such pockets. The bacteria or bacterial enzymes or exotoxins may eventually contact the bone that holds the tooth in place and destroy it.

Treatment and prevention of periodontal disease may include a combination of methods, including, for example, elimination of bacteria causing plaque, reduction of inflammatory processes, stimulation of the immune system, and fortification of the gums. Such treatment and prevention should take place at a physiological pH level, especially considering that oral health is very sensitive to pH, and introducing a pH level higher or lower than normal physiological pH levels to biological tissue could be detrimental to the health of the biological tissue.

Currently there are known methods of treating inflammation of tissue with metals such as copper. For example, it has been known since ancient Egypt that copper has been indicated for therapeutically treating granulomatous inflammation. It has been well established that the dissolution of copper from copper jewelry, for example, bracelets, worn in contact with skin appears to have therapeutic anti-inflammatory effects. In other studies, subdermal copper implants in rats have been demonstrated to exhibit anti-inflammatory activity. In a further instance, a neutral copper (II) bis(glycine) complex perfused through cat skin demonstrating that skin is permeable to soluble copper. In still a further instance several oral and parenteral copper complexes have been somewhat successfully used in the treatment of inflammation or arthritis. Finally, dermally applied copper complexes have been confirmed as pharmacoactive anti-inflammatory agents.

Clearly, various prior art approaches have been taken to employ copper as a means to directly alleviate the causes of inflammation and to promote tissue repair, which has led to have led to several improved copper compositions and dosage forms in an effort to maximize delivery of copper to the inflammatory areas. Examples of such delivery systems of the copper include parenteral (subcutaneous, intravascular, or intramuscular injection), oral, topical or inserts. The parenteral delivery of copper may be painful, inconvenient, require the presence of a physician, and cause further irritation at the site of injection. The oral delivery, on the other hand, often results in poorly absorbed copper by the gastric lining, thereby reducing their anti-inflammatory activity. Finally, the topical delivery of copper is commonly used when selecting a route in medicating inflammation such as, for example, arthritis. The administration of such topical dosage forms are patently desirable because of their unique and advantageous characteristics. otwithstanding the notoriety for topical dosage forms, many past and present topical copper complexes have not performed to their anticipated expectations as a means to effectively and conveniently treat inflammation or arthritis with copper. For example, the application of metal salts to proteinaceous membranes, such as skin, results in the attachment of the copper ions to the membrane components to form copper proteinates or salts. Thus, little if any copper ion, in the soluble, ionized state is ever introduced into the targeted inflammatory, for example, arthritic, areas. Further, copper salts can be corrosive to the skin possibly causing the patient to incur various types of lytic reactions. To overcome this undesirable characteristic, copper ions are complexed with a ligand or chelant to form a metal complex. That is, the copper is shielded from binding to the membrane components. An example of such topical complexes include copper-amine complexes and copper EDTA. Unfortunately, there are undesirable characteristics associated with these complexes which obviate their usefulness.

In U.S. Pat. No. 4,680,309 to the same inventor as the present invention, it is taught that tissue inflammation may be alleviated by delivering a metal complex consisting of a dialaki metal monoheavy metal chelate of an alpha or beta-hydroxy polycarboxlic acid. An example of the metal complex given is dialkalimetal monocopper (II) citrate.

Some individuals have periodontal health issues beyond the typical outlined above. The oral health of individuals undergoing intensive chemotherapy and radiotherapy is further compromised by oral mucositis. Oral mucositis typically starts with a sensation of dry mouth and chapped lips. As symptoms progress, painful whitish patches develop on gums and make it extremely difficult for individuals to eat or drink. Approximately 40% of cancer patients experience oral mucositis, a precursor of often severe periodontitis. According to a December, 2004 issue of The New England Journal of Medicine, no cure or effective treatment is known.

Treatment of oral mucositis should be gentle enough to not cause additional pain to an individual, while also strong enough to treat the symptoms associated with the disorder.

What is desired is an affordable composition for treatment and substantial prevention of periodontal disease that substantially stimulates the immune system and strengthens and promotes healing of gums, while also being within a physiological pH range and able to substantially treat symptoms of oral mucositis.

### SUMMARY

The various exemplary embodiments of the present invention include a composition for treating and preventing periodontal disease. The composition is comprised of acemannan and a hydrated dialkali monometal polycarboxylate 1:1 molar ratio of metal-to-complexing agent.

The various exemplary embodiments further include a method for treating and preventing periodontal disease. The method includes preparing a composition comprising acemannan and a hydrated dialkali monometal polycarboxylate 1:1 molar ratio of metal-to-complexing agent, and introducing the composition into an individual's oral cavity.

### DETAILED DESCRIPTION

The various exemplary embodiments of the present invention comprise an immune system stimulant and an anti-inflammatory agent.

In exemplary embodiments of the present invention, an immune system stimulant is present as acemannan. Acemannan is a complex carbohydrate extract from aloe vera plants, and is considered a primary active component of aloe vera's healing properties. It is believed that acemannan increases the amount of tumor necrosis factor, gamma interferon and interleukin 1, all of which assist in a body's ability to defend and substantially eliminate viruses, bacteria and tumor cells. As a cell nutrient, acemannan has curative properties.

In conjunction with the acemannan, an anti-inflammatory agent may be present as, for example, a hydrated dialkali monometal polycarboxylate 1:1 molar ratio of metal-to-complexing agent such as, for example, disodium monocopper(II) citrate dihydrate (MCC) and related copper complexes.

The metal-to-complexing agent is a multivalent metal and a polyfunctional organic ligand in a ratio of 1:1 of the metal to the ligand and has a dissociation property represented by a sigmoidally shaped plot on a pM-pH diagram. Specific examples of the metal complex are dialkali metal monocopper(II) citrates represented by disodium-, dipotassium- or dilithiummonocopper(II) citrate. These dialkali monocopper(II) citrates have a dissociation property represented by a sigmoidal plot, wherein the curve of two directions meet at a point within the pH range of about 7 to about 9. It has been established that these monocopper(II) complexes in basic media, on the order of about pH 9 to about 12, are very stable, i.e., have an effective stability constant, K_{eff}, of the order of about 10¹² to about 10¹³. However, K_{eff} of these monocopper(II) citrate complexes at a pH of about 7-9 are on the order of about 10⁵ to about 10¹². Therefore, at a pH of around 7, the effective stability constant of the monocopper(II) citrate complex is considerably lower (a thousand to a several hundreds of thousand times lower) and a significant free Cu⁺⁺ concentration is available for anti-inflammatory activity. For example, about 10% of the copper in the complex is in the ionized state at or about pH 7 while approximately 0.1% of the copper is ionized at or about pH 9.

Thus, it is to be understood that the anti-inflammatory complexes of this invention are sensitive to pH, and as the pH is lowered to or below about 7, copper ion is made more available. If tissue is intact, i.e., healthy without trauma, then there are few, if any, free endogenous reacting moieties to induce the dissociation of copper ions. If there is trauma caused by inflammation, then the copper ions are induced to dissociate and complex with the endogenous reacting moieties associated with such trauma, thereby reducing or alleviating the inflammation. In general, the complexes will then tend to dissociate over a pH range of about 3 to about 12. Above about pH 12, the complexes tend to be destroyed by the alkaline media, precipitating from the media as hydrous metal oxides. Below about pH 7, the instability of the metal complex results in high concentrations of the free Cu⁺⁺ upon demand, as explained to effect anti-inflammatory activities. At the pathological pH of about 7, below the skin, the controlled release is most effective. The complexes will preferably be dispersed in a vehicle to provide a composition having a pH of about 6.5 to about 9 for passage through the tissue upon typical administration to provide controlled release of the metal ions upon presentment of endogenous reacting moieties that are associated with inflammatory activities.

In accordance with this description and the presently preferred embodiment, it will become apparent that other metal complexes of polyfunctional organic ligands respond to the model of this invention where they exhibit the dissociation property characterized by a sigmoidal curve on a standard pM-pH diagram. For example, based upon the monometal-polyfunctional organic ligand complex of this invention, other metal ions of a monovalent or multivalent nature, specifically, divalent and polyvalent cations including zinc, nickel, chromium, bismuth, mercury, silver, cobalt, and other similar metallic or heavy metal cations may be employed. Other polyfunctional organic ligands may be substituted for the citric acid specifically exemplified by the preferred embodiment of this invention. Included among other polyfunctional ligands are the broader class of alpha or beta hydroxy polycarboxylic acids into which class the citric acid falls. Also, other functionally substituted acids such as alpha or beta amino, sulfhydro, phosphinol, etc., can be substituted in the molecular model of the metal complex of this invention and similar results can be achieved.

One particularly desirable metal complex in the 1:1 dialkali monometal polyfunctional organic ligand chelate family is disodium monocopper (II) citrate dihydrate, CAS Registry #65330-59-8. This material is sold under the tradename MCC™ by National Research Laboratories, Ltd. of Cincinnati, Ohio.

Most microorganisms are viable around a pH of 7. MCC is advantageous because at a pH between 7 and 9, within physiological pH levels and pH levels for microorganism stability, MCC releases large amounts of toxic metals ions from coordinate structures, thereby denaturizing the cell protein of the microorganisms and causing cell death of the microorganism.

In combination, the acemannan and MCC surprisingly induce an activation of lysyl oxidase, promoting collagen formation and thereby enhancing subsequent tissue repair and regeneration.

In various exemplary embodiments, the acemannan is present in an amount of up to 90% of the weight of the finished product.

In various exemplary embodiments, MCC is present in an effective amount from about 100 mg as copper/liter (about 0.01% w/v) to about 600 mg (about 0.06% w/v) as copper/liter.

The MCC, in addition to activation of lysyl oxidase for collagen formation and subsequent tissue repair, also may serve as a deodorant and an anti-inflammatory agent in the oral cavity.

The composition of the various exemplary embodiments of the present invention may be in the form of a solid, a paste, a gel, a foam or a liquid.

While this invention has been described in conjunction with the specific embodiments outlined above, it is evident that many alternatives, modifications and variations will be apparent to those skilled in the art. Accordingly, the preferred embodiments of the invention as set forth above are intended to be illustrative, not limiting. Various changes may be made without departing from the spirit and scope of the invention.

What is claimed is:

1. A composition for treating and preventing periodontal disease, comprising:
acemannan; and
a hydrated dialkali monometal polycarboxylate 1:1 molar ratio of metal-to-complexing agent.

2. The composition according to claim 1, wherein the hydrated dialkali monometal polycarboxylate 1:1 molar ratio of metal-to-complexing agent is disodium monocopper (II) citrate dihydrate (MCC).

3. The composition according to claim 2, wherein the MCC is present as about 100 mg as copper/liter to about 600 mg as copper/liter.

4. The composition according to claim 1, wherein the acemannan comprises up to 90% of the composition.

5. The composition according to claim 1, wherein the composition is in a form of a solid, a paste, a gel, a foam, or a liquid.

6. A method for treating and preventing periodontal disease, comprising:
preparing a composition comprising acemannan and a hydrated dialkali monometal polycarboxylate 1:1 molar ratio of metal-to-complexing agent; and
introducing the composition into an individual's oral cavity.

7. The method according to claim 6, wherein the hydrated dialkali monometal polycarboxylate 1:1 molar ratio of metal-to-complexing agent is disodium monocopper (II) citrate dihydrate (MCC).

8. The method according to claim 7, wherein the MCC is present as about 100 mg as copper/liter to about 600 mg as copper/liter.

9. The method according to claim 6, wherein the acemannan comprises up to 90% of the composition.

10. The method according to claim 6, wherein the composition is in a form of a solid, a paste, a gel, a foam, or a liquid.
